(19) United States
(12) Plant Patent Application Publication
(10) Pub. No.: US 2002/0144320 P1
(43) Pub. Date: Oct. 3, 2002

(54) Plum tree named "Laetitia"

(76) Inventor: Chris Smith, Stellenbosch (ZA)

Correspondence Address: STRATTON BALLEW PLLC, 213 South 12th Avenue, Yakima, WA, 98902, US

(21) Appl. No.: 09/765,057

(22) Filed: Jan. 16, 2001

(51) Int. Cl. A01H 5/00

(52) U.S. Cl. PLT/184

(57) ABSTRACT

A new and distinct variety of plum (Prunus salicina) named ‘Laetitia’ is disclosed. The new variety originated from an open pollinated seedling of ‘Golden King.’ The tree is semi-erect, sturdy, and vigorous, and appears to be highly resistant to bacterial spot and other leaf diseases. The fruit of the new variety is sweet, attractive, and of good eating quality.

### TECHNICAL FIELD

The invention relates to a new variety of Prunus salicina.

### BRIEF DESCRIPTION OF DRAWINGS

FIG. 1 illustrates the fruit and leaves of the new variety.

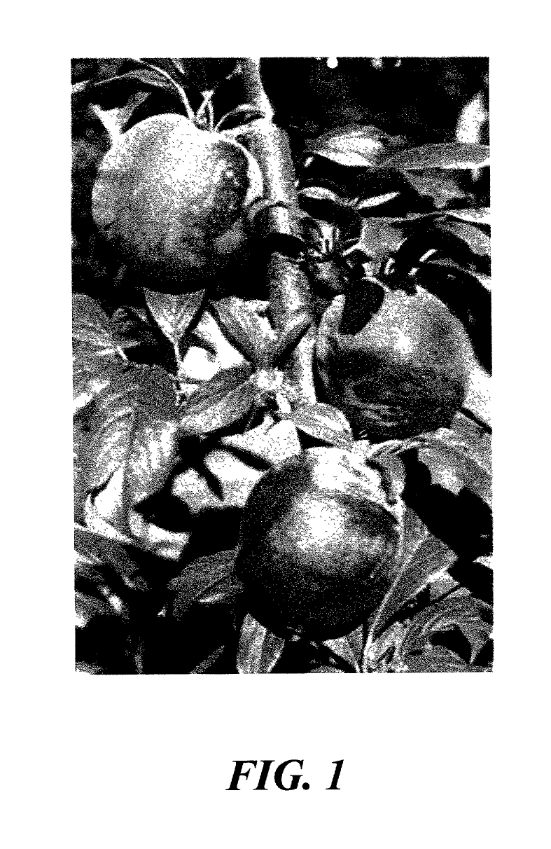

### DESCRIPTION OF THE VARIETY

Origin

Laetitia originated from open pollinated seed of Golden King (unpatented) collected in an orchard where Santa Rosa (unpatented), Gaviota (unpatented) and Red Ace (unpatented) were. The seed was collected in 1972, and the seedlings started bearing in 1977. This seedling was evaluated further at Franschhoek, Steelebosch and Groot Drakenstein.

Tree

The shape of the leaf corresponds remarkably with that of the seed parent, Golden King. The color of Laetitia's leaves, however, is distinctly lighter green than that of the majority of commercial plum cultivars. The tree is semi-erect and sturdy. It is a vigorous grower and apparently is highly resistant to bacterial spot and other leaf diseases.

Laetitia's chilling requirement is lower than that of Songold (unpatented), and no sprays to break dormancy were necessary during the evaluation period.

Blossoming time

Producers must bear in mind that Laetitia is self-incompatible. It flowers at the same time as Songold, Casselmann (unpatented) and Late Santa Rosa (unpatented), which are all good cross-pollinators for it. Very good pollination was obtained in practice by grouping Songold and Laetitia together. This combination also fits in well with regard to orchard practice because the harvesting seasons are close together.

Fruit

The fruit is round-oblong, usually without a point. It has an unobtrusive suture, and small, very shallow stem cavity. The skin surface is smooth, the color bright red and in very exceptional cases, dark purple with a yellowish ground color with quite a number of white lenticels. The flesh color is dark yellow to light orange, fibrous and melting. It has a pleasant, sweet taste and the fragrance is good. When fully ripe, the stone is free from the flesh.

Thinning

With correct cross-pollination, the fruit set is normally very good. One fairly early fruit thinning is sufficient to ensure satisfactory fruit size. Laetitia attains the same size as Songold under identical conditions.

Harvesting

Harvesting at Bien Donne (Republic of South Africa) generally takes place during the third week of January; in other words, 8-10 days prior to Songold. For all practical purposes this cultivar fills the gap between Harry Pickstone (unpatented) and Songold. Harvesting does not last very long.

Marketing

The fruit of Laetitia, like most other plums, can be exported by sea at normal dual temperature storage (−0.5° C. for 10 days, 7.2° C. for approximately 14 days). It has a sweet taste, good eating quality and attractive appearance. Unlike many other plums, Laetitia has already developed a good flavor at the picking ripe stage.

Production

Trees planted in 1980 had already started producing in 1982 and yielded one carton (5 kg) per tree. Production doubled to 10 kg export fruit per tree in 1983, and by 1984 was already 25 kg per tree. Table 1 gives picking dates and size distribution of fruit. Songold trees under similar cultivation practices and of the same age initially produced smaller crops, but in the fourth year its crop equaled that of Laetitia.

TABLE 1
Picking dates and size distribution of crop of Laetitia in
comparison with Harry Pickstone and Songold on two localities.

| | | Size Gradings (percentage of crop) | | | |
|---|---|---|---|---|---|
| Cultivar | Picking Date | 40-45 mm | 45-50 mm | 50-55 mm | 55-60 mm |

At Bien Donne

| | | | | | |
|---|---|---|---|---|---|
| Harry | 12 January | 10 | 65 | 25 | — |
| Pickstone | | | | | |
| Laetitia | 23 January | — | 14 | 20 | 66 |
| Songold | 26 January | — | 12 | 24 | 64 |

At Franschhoek

| | | | | | |
|---|---|---|---|---|---|
| Harry | 14 January | 18 | 72 | 10 | — |
| Pickstone | | | | | |
| Laetitia | 20 January | 5 | 22 | 48 | 25 |
| Songold | 28 January | 7 | 20 | 42 | 31 |

### DETAILED BOTANICAL DESCRIPTION

The following descriptive botanical information was obtained from plants of the new variety grown in the Republic of South Africa.

Flowers:

Time of beginning of flowering.—First week in September.

Length of flowering period.—16 days.

Flower bud—predominant distribution.—Spurs and shoots.

Peduncle length.—7 mm.

Frequency of flowers with double petals.—None or very few.

Flower size.—21 mm.

Position of margins of petals.—Free to touching.

Petal size.—10.4 mm diameter.

Petal shape.—Round to obovate.

Undulation of petal margin.—Weak to medium.

Sepal shape.—Ovate.

Position of stigma compared to anthers.—Below to same level.

Leaves:

Leaf: attitude.—Horizontal.

Leaf blade: shape.—Broad obovate.

Leaf: angle of the apex.—Right angle or nearly right angle.

Leaf: glossiness of upper side.—Medium.

Leaf blade: pubescence on lower side.—Weak.

Leaf blade: incisions of margin.—Crenate.

Petiole: pubescence on upper side.—Absent or very weak.

Petiole: depth of groove.—Medium

Petiole: anthocyanin coloration of upper side.—Weak.

Petiole: anthocyanin coloration of lower side.—Absent.

Glands.—Present.

Petiole: number of glands.—0.1.

Leaf base: number of glands.—0.5.

Leaf: position of glands.—On both leaf base and petiole.

Leaf blade: avg. length.—90.5 mm.

Leaf blade: avg. width.—40.8 mm.

Leaf area.—3696.

Leaf: ratio length/width.—2.2.

Petiole: avg. length.—15.7 mm.

Fruit:

Time of ripening.—Middle to end of January.

Length of period for development of fruit.—128 days.

Position of maximum diameter.—At center.

Symmetry (front view).—Symmetrical.

Shape of apex.—Flat, sometimes with slight point.

Depth of stalk cavity.—7.3 mm.

Ground color of skin (wax coat included).—Yellow.

Over color of skin (wax coat included).—Red.

Extent of over color coloration.—61% to whole fruit covered.

Type of over color.—Solid flash to small flecks.

Length.—56.5 mm.

Width.—54.2 mm.

Size.—55.4.

General shape (profile view).—Rounded to oblong.

Color of flesh.—Yellow.

Firmness of flesh.—Firm.

Juiciness.—Medium.

Acidity.—Medium.

Sweetness.—14 Brix.

Adherence of stone to flesh.—Semi-adherent.

Stone:

Length.—23.1 mm.

Width in frontal view.—9.3 mm.

Width in profile view.—15.8 mm.

Size.—368.

Shape in profile view.—Long-elliptical.

Shape in ventral view.—Sub-globular.

Shape in basal view.—Elliptical.

Symmetry in profile view.—Asymmetrical.

Symmetry in ventral view.—Symmetrical.

Position of maximum width (ventral view).—At center.

Texture of lateral surfaces.—Medium granular to rough.

Dorsal groove.—Broken.

Sharpness of edges.—Medium.

Width of ventral zone.—Small.

Width of stalk end.—Medium.

Angle of stalk end.—Right angle or nearly right angle.

Shape of pistil end.—Intermediate.

Autumn Leaf Fall & 1-year old shoot:

Autumn leaf fall.—Middle June.

Autumn leaf color.—Green yellow.

Tree vigor.—strong.

Tree growth habit.—Semi-erect.

Tree density.—medium.

1 Year shoot: attitude.—Semi-erect.

1 Year shoot: intensity of color (sun side, after removal of cuticle).—Medium.

1 Year shoot: intensity of color (opposite sun side, after removal of cuticle).—Light to medium.

Tree: number of spurs.—Medium.

Spur: length.—Short to medium.

Wood bud: size.—Medium.

Wood bud: shape.—Conical.

Wood bud: position relative to shoot.—Markedly held out.

What is claimed is:

1. A new and distinct variety of Prunus salicina, as illustrated and described herein.
